(19) United States
(12) Patent Application Publication
(10) Pub. No.: US 2022/0326268 A1
(43) Pub. Date: Oct. 13, 2022

(54) METHOD FOR AUTOMATICALLY DISTINGUISHING WHOLE BLOOD/PLASMA/NON-SUCTION BY MEANS OF REFLECTIVE PHOTOSENSOR

(71) Applicant: I-SENS, INC., Seoul (KR)

(72) Inventors: Jungmin YOU, Seoul (KR); Jae Won JUNG, Seoul (KR); Yungjoon JIN, Seoul (KR); Yu Sung KIM, Seoul (KR); Ui Sik KIM, Seoul (KR); Woo Seob JIN, Seoul (KR); Miin HONG, Seoul (KR)

(21) Appl. No.: 17/597,247

(22) PCT Filed: Jan. 15, 2021
(86) PCT No.: PCT/KR2021/000611

§ 371 Date: Dec. 30, 2021

(30) Foreign Application Priority Data
Feb. 10, 2020 (KR) 10-2020-0015762

(51) Int. Cl. G01N 35/10; G01N 35/00; G01N 33/543; G01N 21/31; G01N 1/40

(52) U.S. Cl. CPC G01N 35/1009; G01N 35/00623; G01N 33/54366; G01N 21/31; G01N 1/4077; G01N 2035/1034; G01N 2035/103; G01N 2201/062; G01N 2001/4083

(57) ABSTRACT

A method of automatically distinguishing absorption or non-absorption of whole blood or blood plasma by using a reflective photosensor in an automatic immunoassay device including a round cartridge which may simultaneously perform the centrifugation and automatic analysis of a blood sample and a tip which may be moved up, down, left and right based on the round cartridge, the method includes: installing the reflective photosensor below the round cartridge; mounting the tip above the round cartridge, and continuously measuring blood non-absorption data in a range including a position where the blood is absorbed by using the reflective photosensor, collecting the measured data and storing the collected data while the tip is raised; continuously measuring blood absorption data by using the reflective photosensor while the tip is lowered and absorbs the blood present in the range including the position where the blood is absorbed from the round cartridge; and determining whether a type of the blood is whole blood or blood plasma, or whether the whole blood or the blood plasma is not absorbed by comparing the blood non-absorption data with the blood absorption data.

### TECHNICAL FIELD

The present invention relates to a method of automatically distinguishing absorption or non-absorption of whole blood or blood plasma by using a reflective photosensor, and more particularly, to a method of automatically distinguishing whether whole blood or blood plasma is absorbed or whether the whole blood or the blood plasma is not absorbed by using a reflective photosensor, the method being capable of accurately checking whether the whole blood or the blood plasma is absorbed or whether the whole blood or the blood plasma is not absorbed from a round cartridge into a pipette mounted on a tip of an automatic immunoassay device including the round cartridge which may simultaneously perform the centrifugation and automatic analysis of a blood sample and the tip which may be moved up, down, left and right based on the round cartridge.

### BACKGROUND ART

In general, an immunoassay device may measure concentration of a specific biomarker protein in the blood plasma or serum of a human body.

The immunoassay device is not necessary to calibrate the measured concentration before outputting a result when directly using the blood plasma or serum as a measurement sample. However, when using whole blood as the measurement sample and analyzing the blood plasma or serum in the whole blood, the immunoassay device is necessary to correct the measured concentration by using a hematocrit of the measurement sample before outputting the result.

In general, a conventional immunoassay device may require centrifugation of the measurement sample separately performed in an external laboratory, or the manually input measured hematocrit and type (whole blood/blood plasma/serum) of the sample.

In this case, it may be necessary to separately input information on the measurement sample.

In addition, in order to solve this problem, a method has been proposed in which the hematocrit is optically measured to correct the result. However, it is impossible to generally use this method for all the immunoassay devices.

In addition, it may be inconvenient to separately input the information on the measurement sample.

That is, when using the sample of the whole blood, it is necessary to calculate and correct a ratio of a volume occupied by red blood cells to a total volume of the whole blood, i.e. the hematocrit, as a parameter to be considered when analyzing a substance included in a blood plasma component, and this parameter may significantly depend on age, climate, nutrition, illness and another factor. For example, a hematocrit of 40% may indicate that the red blood cells occupy 40% of the volume of the whole blood and the blood plasmas occupy 60% thereof. Therefore, when a volume of the hematocrit is increased in a patient's blood, a volume of the blood plasma may be decreased in the blood sample having a predetermined constant volume which is injected into the test device, and vice versa.

Only the blood plasma component may contain an analyte to be measured. Therefore, when a reaction mixture gets a smaller volume of the blood plasma component added thereto, the reaction mixture may have a reduced concentration of the substance to be measured therein, thus obtaining a smaller analytical value, and vice versa.

In any and every analysis of the concentration of the blood plasma substance in whole blood, it is necessary to perform a correction for a change in the hematocrit to obtain a true blood plasma concentration.

In order to omit this correction, it is possible to perform an analysis on serum or blood plasma in which the red blood cells are filtered or centrifuged in advance, which may increase the cost due to a complex design. In addition, most of the immunoassay devices may require the manual input of the whole blood, blood plasma or serum, which may not only be very cumbersome for an analyst but also may significantly reduce accuracy of the immunoassay if the analyst makes an error.

### DISCLOSURE

### Technical Problem

An exemplary embodiment of the present invention provides to a method of a method of automatically distinguishing absorption or non-absorption of whole blood or blood plasma by using a reflective photosensor, the method being capable of accurately checking whether the whole blood or the blood plasma is absorbed or whether the whole blood or the blood plasma is not absorbed into a pipette mounted on a tip of an automatic immunoassay device including a round cartridge which may simultaneously perform the centrifugation and automatic analysis of a blood sample and the tip which may be moved up, down, left and right based on the round cartridge.

### Technical Solution

According to an exemplary embodiment of the present invention, a method of automatically distinguishing absorption or non-absorption of whole blood or blood plasma by using a reflective photosensor in an automatic immunoassay device including a round cartridge which may simultaneously perform the centrifugation and automatic analysis of a blood sample and a tip which may be moved up, down, left and right based on the round cartridge, the method includes: installing the reflective photosensor below the round cartridge; mounting the tip above the round cartridge, and continuously measuring blood non-absorption data in a range including a position where the blood is absorbed by using the reflective photosensor, collecting the measured data and storing the collected data while the tip is raised;

continuously measuring blood absorption data by using the reflective photosensor while the tip is lowered and absorbs the blood present in the range including the position where the blood is absorbed from the round cartridge; and determining whether a type of the blood is whole blood or blood plasma, or whether the whole blood or the blood plasma is not absorbed by comparing the blood non-absorption data with the blood absorption data.

### ADVANTAGEOUS EFFECTS

As set forth above, the method of automatically distinguishing the absorption or non-absorption of the whole blood or the blood plasma by using the reflective photosensor according to an exemplary embodiment of the present invention may accurately check whether the whole blood or the blood plasma is absorbed or whether the whole blood or the blood plasma is not absorbed from the round cartridge into the pipette mounted on the tip of the automatic immunoassay device including the round cartridge which may simultaneously perform the centrifugation and automatic analysis of the blood sample and the tip which may be moved up, down, left and right based on the round cartridge.

### DESCRIPTION OF THE DRAWINGS

FIG. 1 is a side view of an automatic immunoassay device including a tip which may be moved up, down, left and right based on a conventional round cartridge.

FIGS. 2 and 3 are a graph showing a method of determining whether a type of the blood is whole blood or blood plasma or whether the whole blood or the blood plasma is not absorbed, by comparing blood non-absorption data with blood absorption data in the automatic immunoassay device including the tip which may be moved up, down, left and right based on the conventional round cartridge, and a graph showing a determination result value obtained from the same tip used for one measurement.

FIG. 4 is a flowchart showing a method of comparing the blood non-absorption data with the blood absorption data in the automatic immunoassay device including the tip which may be moved up, down, left and right based on a round cartridge according to an exemplary embodiment of the present invention to determine whether the type of the blood is the whole blood or the blood plasma, or whether the whole blood or the blood plasma is not absorbed.

FIG. 5 is a side view showing an operation of the automatic immunoassay device including the tip which may be moved up, down, left and right based on the round cartridge according to an exemplary embodiment of the present invention.

FIG. 6 is a graph showing a method of determining whether the type of the blood is the whole blood or the blood plasma, or whether the whole blood or the blood plasma is not absorbed, by comparing the blood non-absorption data with the blood absorption data in the automatic immunoassay device including the tip which may be moved up, down, left and right based on the round cartridge according to an exemplary embodiment of the present invention.

FIGS. 7 to 10 are graphs showing whether the type of the blood is the whole blood or the blood plasma and a result value of the whole blood or blood plasma when the whole blood or the blood plasma is not absorbed by comparing the blood non-absorption data with the blood absorption data, obtained from the same tip used for one measurement, in the automatic immunoassay device including the tip which may be moved up, down, left and right based on the round cartridge according to an exemplary embodiment of the present invention.

FIGS. 11 and 12 are graphs showing whether the type of the blood is the whole blood or the blood plasma and the result value of the whole blood or blood plasma when the whole blood or the blood plasma is not absorbed by comparing the blood non-absorption data with the blood absorption data, obtained from two different tips used for one measurement, in the automatic immunoassay device including the tip which may be moved up, down, left and right based on the round cartridge according to an exemplary embodiment of the present invention.

### MODE FOR INVENTION

Hereinafter, exemplary embodiments of the present invention will be described in detail with reference to the accompanying drawings so that those skilled in the art to which the present invention pertains may easily practice the present invention. The present invention may be modified in various different forms, and is not limited to embodiments provided in the present specification. In addition, in the drawings, portions unrelated to the description are omitted to clearly describe the present invention, and similar portions are denoted by similar reference numerals throughout the specification.

In the specification, unless explicitly described to the contrary,“including” any component will be understood to imply the inclusion of other components rather than the exclusion of any other components. In addition, a term “part,” “module,” “device” or the like, described in the specification may indicate a unit of processing at least one function or operation, which may be implemented by hardware or software or a combination of hardware and software.

Hereinafter, the description describes an automatic analysis device including a cartridge capable of performing the centrifugation and automatic analysis of a blood sample, according to an exemplary embodiment of the present invention, in detail with reference to the attached drawing.

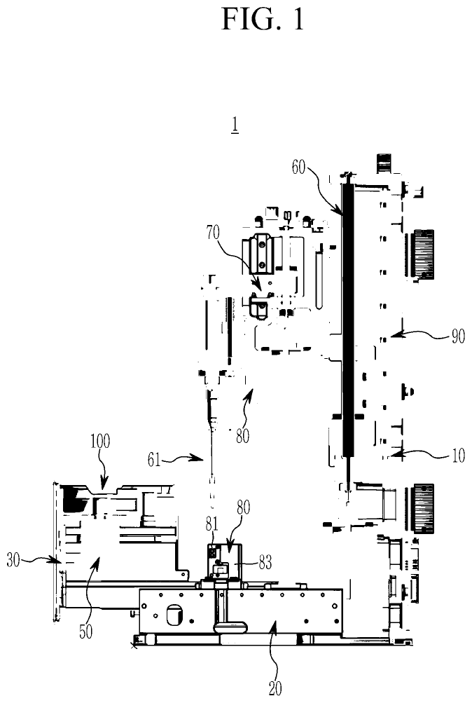

In order to solve problems of the prior art, as shown in FIG. 1, Korean Patent Publication No. 10-1970790, entitled “Method and apparatus with improved accuracy”, which is filed and registered by the same applicant, shows an automatic analysis device 1 including a cartridge 100 capable of performing the centrifugation and automatic analysis of the blood sample, in which the cartridge 100 capable of performing the centrifugation and automatic analysis of the blood is installed in a cartridge accommodation housing 30 by reciprocating a main frame 10 disposed at a bottom of a cartridge accommodation housing 30 to an X-axis transfer unit 20 in an X-axis direction, a sample is injected into the cartridge 100 by moving a tip 61 by a tip elevation unit 60 disposed at an upper portion of the device 1, the cartridge is rotated by a rotary drive unit 50 disposed at a lower portion of the device 1 to centrifuge whole blood or rotated to repeatedly absorb/discharge a reagent, and an analysis process is performed by using a measurement unit 70.

Here, the main frame 10 may further include a reflective photosensor 80 accommodated in the cartridge accommodation housing 30 and automatically measuring, from the tip 61, whether a type of the blood is the whole blood or the blood plasma, or whether the whole blood or the blood plasma is not absorbed. The reflective photosensor 80 may include a light source 81 and a light receiving element 83. The light source 81 may preferably be a long and thin light emitting diode (LED) disposed in an axial direction of the cartridge 100, and the light receiving element 83 may preferably be a charge coupled device (CCD) linear image sensor in which the plurality of light receiving elements are arranged in one row in the axial direction. The light receiving element 83 may receive light reflected from the light source 81 on a well surface of the cartridge 100.

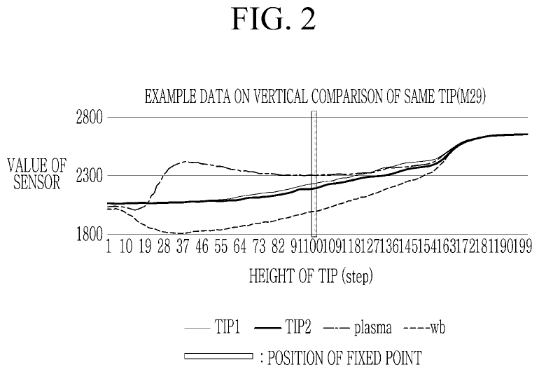

Here, as shown in FIGS. 2 to 4, when the tip 61 reaches a height of 100 steps, which is half of a total height of 200 steps, i.e., when the tip 61 reaches its fixed point, it is possible to irradiate light from the light source 81 of the reflective photosensor 80, and read an output value of the light receiving sensor 83 reflected from a detected substance. It is then possible to compare a result value obtained from the same tip 61 used for one measurement or different tips 61, with a correction value of whole blood (wb) or the blood plasma to determine whether the type of the blood is the whole blood or the blood plasma, or whether the whole blood or the blood plasma is absorbed.

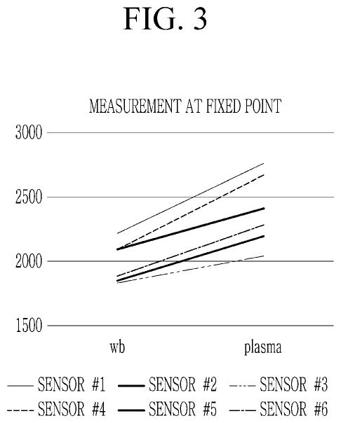

However, the above-described method of reading the output value of the reflective photo sensor 80 at the fixed point of the tip 61 by using the sensor and comparing the output values of the whole blood and blood plasma with their correction values to make the determination may be difficult to accurately distinguish the values from each other due to deviation in device performance of the light receiving sensor 83 when using the different light receiving sensor 83, as shown in FIG. 3 and Table 1, and may be unable to accurately distinguish the two values from each other because the obtained output values may have an insufficient margin therebetween.

TABLE 1

| Sensor no. | Wb absorbed | Plasma absorbed |
|---|---|---|
| 1 | 2221 | 2274 |
| 2 | 1862 | 2202 |
| 3 | 1840 | 2044 |
| 4 | 2092 | 2679 |
| 5 | 2092 | 2422 |
| 6 | 1906 | 2291 |

As shown in Table 1 and FIG. 3, the light receiving sensors 83, i.e. sensor nos. 1, 2, 3, 4, 5 and 6, may each have a higher measurement value obtained when the whole blood (wb) is absorbed than a measurement value obtained when the blood plasma is absorbed. However, some sensors may show the measurement value of the whole blood (wb) is the same as the measurement value of the blood plasma (Blood plasma). In this case, the two values may not be clearly distinguished from each other.

In addition, each correction value of the two measurement values is necessary to be pre-determined for performing one measurement, depending on whether the type of the blood is the whole blood or the blood plasma whenever measuring the whole blood or the blood plasma is not absorbed into the tip 61.

In addition, the tip is necessary to be replaced for every measurement, and the measurement value may thus also be changed.

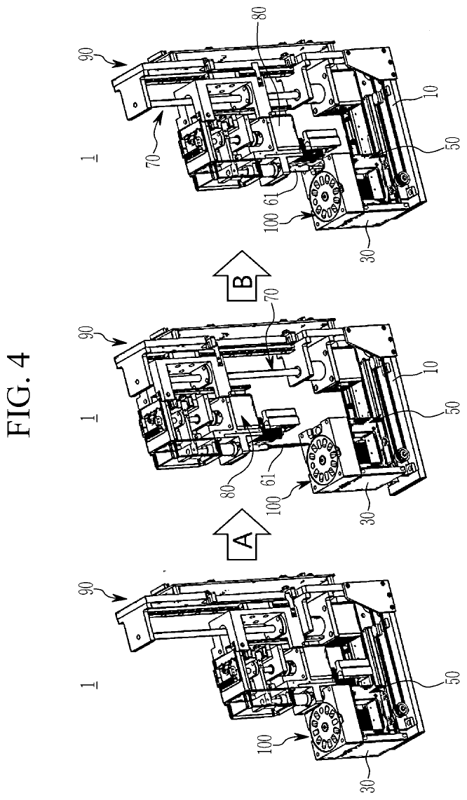

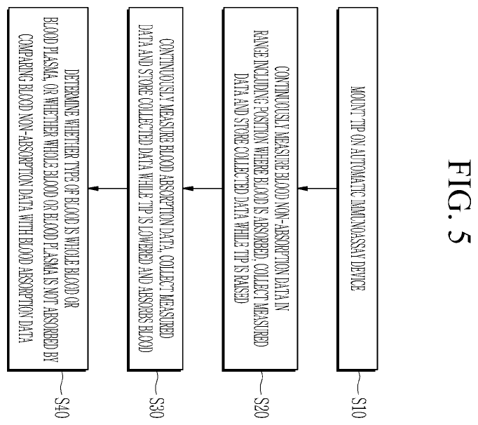

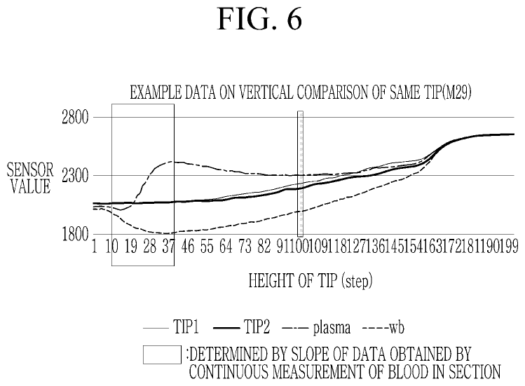

Hereinafter, the description describes a method of automatically distinguishing absorption or non-absorption of whole blood or blood plasma by using a reflective photosensor according to an exemplary embodiment of the present invention in detail with reference to FIGS. 4 and 5.

FIG. 4 is a flowchart showing a method of comparing the blood non-absorption data with blood absorption data in the automatic immunoassay device including the tip which may be moved up, down, left and right based on a round cartridge according to an exemplary embodiment of the present invention to determine whether the type of the blood is the whole blood or the blood plasma, or whether the whole blood or the blood plasma is not absorbed; and FIG. 5 is a side view showing an operation of the automatic immunoassay device including the tip which may be moved up, down, left and right based on the round cartridge according to an exemplary embodiment of the present invention.

As shown in FIGS. 4 and 5, the method of automatically distinguishing absorption or non-absorption of whole blood or blood plasma by using a reflective photosensor according to an exemplary embodiment of the present invention may provide a constant measurement value regardless of the device performance of the light receiving sensor 83, may clearly distinguish the measurement values of the whole blood and blood plasma because the difference between the measurement values of the whole blood and blood plasma is significant, and may have no need to set the respective correction values thereof in advance based on whether the whole blood or the blood plasma is absorbed or not absorbed.

The method of automatically distinguishing absorption or non-absorption of whole blood or blood plasma by using a reflective photosensor according to an exemplary embodiment of the present invention, which is improved for this purpose, in the automatic immunoassay device 1 including the round cartridge 100 which may simultaneously perform the centrifugation and automatic analysis of the blood sample and the tip which may be moved up, down, left and right based on the round cartridge, may include: installing the reflective photosensor 80 below the round cartridge 100 and mounting the tip 61 above the round cartridge 100 (S10), and continuously measuring the blood non-absorption data in a range including a position where the blood is absorbed by using the reflective photosensor, collecting the measured data and storing the collected data while the tip 61 is raised (S20).

The method may include continuously measuring the blood absorption data by using the reflective photosensor while the tip is lowered and absorbs the blood present in the range including the position where the blood is absorbed from the round cartridge, collecting the measured data and storing the collected data (S30).

The method may include determining whether the type of the blood is the whole blood or the blood plasma, or whether the whole blood or the blood plasma is not absorbed by comparing the blood non-absorption data with the blood absorption data (S40).

Here, the light source 81 of the reflective photosensor 80 may be the long and thin light emitting diode (LED) disposed below the cartridge 100 in the axial direction of the cartridge 100, and the light receiving element 83 may be the charge coupled device (CCD) linear image sensor disposed opposite to the light source 81, and installed in one row below the cartridge 100 in the axial direction of the cartridge 100.

In order to determine whether the type of the blood is the whole blood or the blood plasma, or whether the whole blood or the blood plasma is not absorbed by comparing the blood non-absorption data with the blood absorption data, several measurements may be continuously performed to measure whether the whole blood or the blood plasma is not absorbed into the same tip 61 used for one measurement, whether the whole blood or the blood plasma is not absorbed into at least two or more different tips 61 used for different measurements, and whether the whole blood is absorbed into the tip 61 and whether the blood plasma is absorbed into the tip 61.

In order to determine whether the type of the blood is the whole blood or the blood plasma, or whether the whole blood or the blood plasma is not absorbed by comparing the blood non-absorption data with the blood absorption data, the method of automatically distinguishing absorption or non-absorption of whole blood or blood plasma by using a reflective photosensor according to an exemplary embodiment of the present invention may use a slope of data obtained from the continuous measurements of the blood in a specific section.

Therefore, it is possible to reduce the deviation by using the slope of the data obtained from the continuous measurements of the blood rather than one fixed measurement value.

As shown in FIG. 5, the slope may be a relative ratio of the measurement value of the whole blood or blood plasma obtained by using the reflective photosensor 80 to the height of the tip 61.

The specific section may be a section including first and second inflection points of the values obtained from the continuous measurements of whether the whole blood (wb) and the (blood) plasma by using the reflective photosensor 80, i.e., the point at which a difference between the values obtained from the continuous measurements of the whole blood (wb) and the (blood) plasma by using the reflective photosensor 80 begins to occur and the point at which the slope of the values obtained from the continuous measurements of the whole blood (wb) and the (blood) plasma by using the reflective photosensor 80 has a maximum value.

In the specific section, the tip 61 may be disposed at a height of 10 steps or more and 46 steps or less when the total height is 200 steps, and 500 to 700 may be the maximum value of the difference between the measurement values of the whole blood and the blood plasma obtained by using the reflective photosensor.

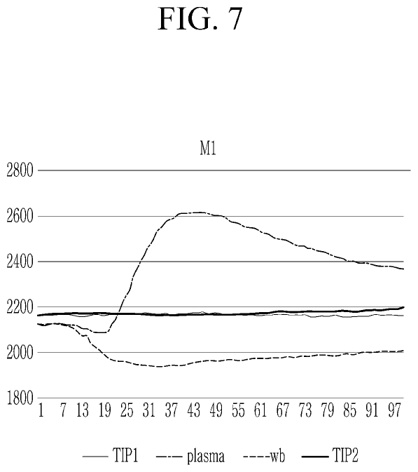

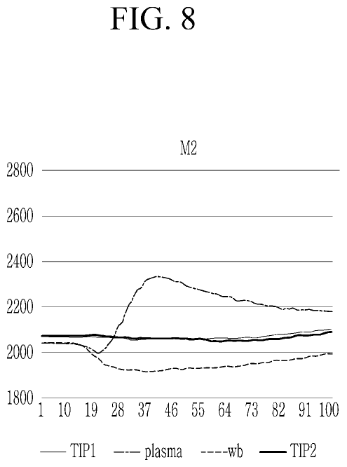

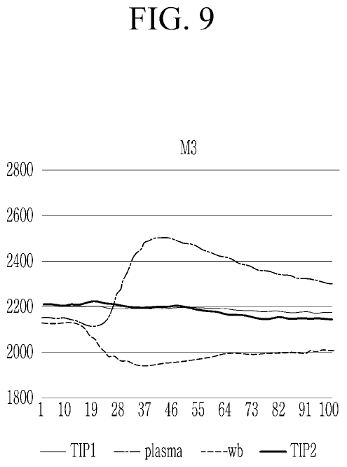

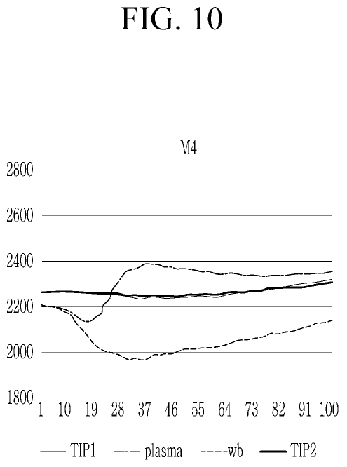

Hereinafter, referring to FIGS. 7 through 12 and Tables 1-2 and 2-2, the description describes that the method of automatically distinguishing absorption or non-absorption of whole blood or blood plasma by using a reflective photosensor according to an exemplary embodiment of the present invention may provide the constant measurement value regardless of the device performance of the light receiving sensor 83, may clearly distinguish the measurement values of the whole blood and blood plasma because the difference between the measurement values of the whole blood and blood plasma is significant, and may have no need to set the respective correction values thereof in advance based on whether the whole blood or the blood plasma is absorbed or not absorbed.

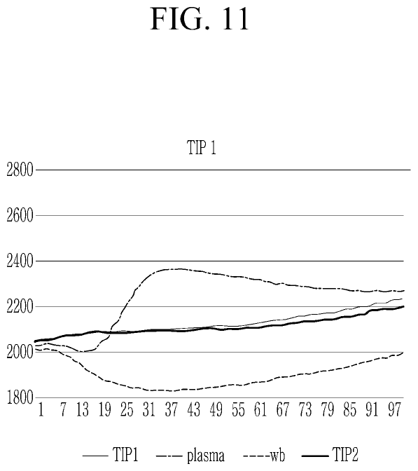

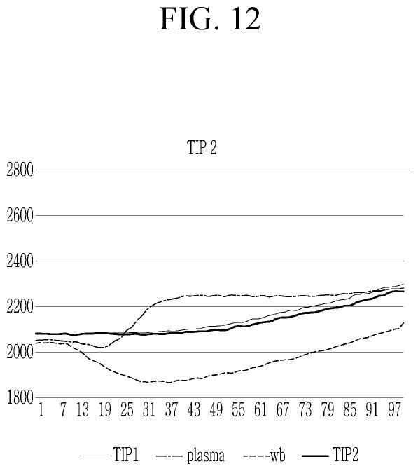

FIGS. 7 to 10 are graphs showing whether the type of blood is the whole blood or the blood plasma and a result value of the whole blood or the blood plasma when the whole blood or the blood plasma is not absorbed by comparing the blood non-absorption data with the blood absorption data, obtained from the same tip used for one measurement, in the automatic immunoassay device including the tip which may be moved up, down, left and right based on the round cartridge according to an exemplary embodiment of the present invention; and FIGS. 11 and 12 are graphs showing whether the type of the blood is the whole blood or the blood plasma and the result value of the whole blood or the blood plasma when the whole blood or the blood plasma is not absorbed by comparing the blood non-absorption data with the blood absorption data, obtained from two different tips used for one measurement, in the automatic immunoassay device including the tip which may be moved up, down, left and right based on the round cartridge according to an exemplary embodiment of the present invention.

As shown in FIGS. 7 to 10, according to the method of automatically distinguishing absorption or non-absorption of whole blood or blood plasma by using a reflective photosensor according to an exemplary embodiment of the present invention, the values continuously measured by the four light receiving sensors 83, i.e. sensors M1, M2, M3 and M4, and the graphs of the values all show the same pattern.

That is, the method may provide the constant measurement value regardless of the device performance of the light receiving sensor 83, and may clearly distinguish the measurement values of the whole blood and blood plasma because the difference between the measurement values of the whole blood and blood plasma is significant.

TABLE 2-1

| | M1 | | | | M2 | | | | M3 | | | | M4 | | | |
|---|---|---|---|---|---|---|---|---|---|---|---|---|---|---|---|---|
| | tip | plsam | wb | tip2 | tip | plsam | wb | tip2 | tip | plsam | wb | tip2 | tip | wb | wb | tip2 |
| 0 | 2164 | 2122 | 2123 | 2155 | 2074 | 2043 | 2045 | 2077 | 2204 | 2151 | 2120 | 2207 | 2270 | 2215 | 2206 | 2274 |
| 1 | 2163 | 2118 | 2125 | 2161 | 2071 | 2042 | 2046 | 2076 | 2204 | 2153 | 2120 | 2205 | 2270 | 2208 | 2206 | 2276 |
| 2 | 2165 | 2122 | 2123 | 2162 | 2069 | 2042 | 2045 | 2075 | 2205 | 2153 | 2122 | 2206 | 2269 | 2210 | 2206 | 2274 |
| 3 | 2164 | 2121 | 2123 | 2161 | 2070 | 2043 | 2046 | 2076 | 2204 | 2150 | 2122 | 2207 | 2270 | 2209 | 2207 | 2274 |
| 4 | 2168 | 2123 | 2122 | 2163 | 2070 | 2040 | 2047 | 2075 | 2204 | 2149 | 2123 | 2206 | 2268 | 2207 | 2204 | 2273 |
| 5 | 2160 | 2122 | 2122 | 2164 | 2067 | 2043 | 2046 | 2074 | 2201 | 2151 | 2123 | 2206 | 2270 | 2207 | 2202 | 2274 |
| 6 | 2166 | 2121 | 2120 | 2165 | 2067 | 2043 | 2044 | 2077 | 2203 | 2149 | 2127 | 2207 | 2269 | 2203 | 2200 | 2277 |
| 7 | 2166 | 2122 | 2119 | 2166 | 2069 | 2042 | 2045 | 2077 | 2201 | 2142 | 2128 | 2203 | 2270 | 2202 | 2295 | 2274 |
| 8 | 2168 | 2218 | 2117 | 2166 | 2068 | 2044 | 2046 | 2075 | 2203 | 2146 | 2130 | 2207 | 2271 | 2199 | 2191 | 2274 |
| 9 | 2166 | 2118 | 2108 | 2164 | 2069 | 2040 | 2044 | 2074 | 2203 | 2144 | 2128 | 2207 | 2221 | 2195 | 2181 | 2274 |
| 10 | 2168 | 2115 | 2103 | 2161 | 2071 | 2041 | 2041 | 2075 | 2202 | 2137 | 2127 | 2204 | 2273 | 2186 | 2177 | 2276 |
| 11 | 2168 | 2108 | 2091 | 2159 | 2070 | 2038 | 2036 | 2073 | 2201 | 2136 | 2124 | 2207 | 2271 | 2182 | 2159 | 2275 |
| 12 | 2167 | 2102 | 2071 | 2157 | 2071 | 2031 | 2030 | 2074 | 2201 | 2131 | 2116 | 2209 | 2271 | 2170 | 2139 | 2277 |
| 13 | 2169 | 2100 | 2070 | 2159 | 2069 | 2024 | 2017 | 2075 | 2200 | 2125 | 2107 | 2213 | 2271 | 2186 | 2128 | 2276 |
| 14 | 2169 | 2090 | 2039 | 2160 | 2070 | 2018 | 2011 | 2075 | 2199 | 2123 | 2100 | 2217 | 2273 | 2151 | 2108 | 2276 |
| 15 | 2171 | 2086 | 2021 | 2162 | 2069 | 2009 | 2050 | 2075 | 2199 | 2118 | 2084 | 2220 | 2272 | 2144 | 2090 | 2277 |
| 16 | 2169 | 2082 | 2012 | 21 [symbol] [symbol] | 2068 | 2003 | 1986 | 2074 | 2198 | 2115 | 2062 | 2220 | 2271 | 2141 | 2081 | 2273 |
| 17 | 2169 | 2083 | 1997 | 2162 | 2086 | 2002 | 1978 | 2072 | 2197 | 2114 | 2056 | 2220 | 2267 | 2145 | 2065 | 2274 |
| 18 | 2167 | 2087 | 1985 | 2162 | 2068 | 2003 | 1968 | 2071 | 2197 | 2115 | 2031 | 2214 | 2265 | 2155 | 2052 | 2272 |
| 19 | 2166 | 2103 | 1978 | 2163 | 2065 | 2010 | 1958 | 2072 | 2196 | 2122 | 2013 | 2212 | 2264 | 2175 | 2046 | 2270 |
| 20 | 2166 | 2132 | 1970 | 2162 | 2065 | 2016 | 1952 | 2072 | 2194 | 2128 | 2007 | 2211 | 226 [symbol] | 2184 | 2036 | 2268 |
| 21 | 2166 | 2151 | 1967 | 2164 | 2066 | 2031 | 1945 | 2071 | 2195 | 2146 | 1994 | 2207 | 2259 | 2208 | 2027 | 2267 |
| 22 | 216 [symbol] | 2194 | 1965 | 2165 | 2064 | 2053 | 1938 | 2069 | 2193 | 2175 | 1987 | 2205 | 2261 | 2233 | 2021 | 2265 |
| 23 | 2167 | 2239 | 1959 | 2164 | 2065 | 2066 | 1936 | 2068 | 2192 | 2190 | 1990 | 2203 | 2259 | 2246 | 2016 | 2265 |
| 24 | 2165 | 2267 | 1956 | 2168 | 2063 | 2095 | 1933 | 2068 | 2194 | 2225 | 1975 | 2201 | 2260 | 2272 | 2009 | 2265 |
| 25 | 2168 | 2314 | 1953 | 2167 | 2065 | 2125 | 1930 | 2068 | 2192 | 2263 | 1969 | 2200 | 2262 | 2299 | 2007 | 2267 |
| 26 | 2168 | 2358 | 1950 | 2169 | 2061 | 2141 | 1928 | 2067 | 2191 | 2280 | 1965 | 2200 | 2261 | 2309 | 2002 | 2266 |
| 27 | 2168 | 2379 | 1945 | 2168 | 2061 | 2173 | 1924 | 2065 | 2193 | 2322 | 1957 | 2200 | 2261 | 2330 | 1996 | 2264 |
| 28 | 2167 | 2418 | 1944 | 2170 | 2050 | 2206 | 1922 | 2007 | 2191 | 2356 | 1955 | 2196 | 2258 | 3247 | 1994 | 2264 |
| 29 | 2166 | 2453 | 1940 | 2167 | 2058 | 2222 | 1921 | 2065 | 2190 | 2373 | 1951 | 2195 | 2256 | 2354 | 1991 | 2287 |
| 30 | 2165 | 2472 | 1939 | 2169 | 2057 | 2251 | 1919 | 2062 | 2189 | 2403 | 1949 | 2194 | 2251 | 2371 | 1986 | 2256 |
| 31 | 2164 | 2503 | 1939 | 2166 | 2058 | 2778 | 1918 | 2064 | 2188 | 2427 | 1944 | 2191 | 2251 | 2374 | 1984 | 2260 |
| 32 | 2162 | 2531 | 1939 | 2165 | 2060 | 2287 | 1916 | 2062 | 2186 | 2439 | 1946 | 2190 | 2251 | 2377 | 1987 | 2255 |
| 33 | 2162 | 2544 | 1940 | 2166 | 2057 | 2307 | 1917 | 2063 | 2189 | 2458 | 1945 | 2190 | 2245 | 2382 | 1985 | 2257 |
| 34 | 2164 | 2562 | 1940 | 2168 | 2058 | 2321 | 1915 | 2064 | 2191 | 2473 | 1946 | 2193 | 2251 | 2384 | 1982 | 2257 |
| 35 | 2161 | 2580 | 1945 | 2164 | 2059 | 2328 | 1914 | 2064 | 2191 | 2478 | 1948 | 2197 | 2253 | 2389 | 1990 | 2260 |
| 36 | 2162 | 2582 | 1946 | 2166 | 2061 | 2334 | 1917 | 2063 | 2192 | 2487 | 1949 | 2193 | 2253 | 2391 | 1993 | 2258 |
| 37 | 2163 | 2592 | 1946 | 2164 | 2060 | 2339 | 1919 | 2066 | 2190 | 2490 | 1953 | 2197 | 2254 | 2390 | 1994 | 2257 |
| 38 | 2165 | 2602 | 1950 | 2168 | 2060 | 2341 | 1919 | 2063 | 2193 | 2493 | 1955 | 2197 | 2253 | 2390 | 1999 | 2259 |
| 39 | 2165 | 2602 | 1955 | 2169 | 2058 | 2340 | 1918 | 2064 | 2194 | 2493 | 1951 | 2196 | 2253 | 2389 | 2002 | 2257 |
| 40 | 2164 | 2606 | 1954 | 2170 | 2059 | 2340 | 1920 | 2064 | 2194 | 2495 | 1956 | 2195 | 2251 | 2386 | 2000 | 2258 |
| 41 | 2164 | 2607 | 1957 | 2168 | 2058 | 2336 | 1921 | 2062 | 2196 | 2493 | 1958 | 2198 | 2250 | 2387 | 2005 | 2257 |
| 42 | 2165 | 2604 | 1960 | 2169 | 2059 | 2333 | 1922 | 2063 | 2196 | 2491 | 1960 | 2196 | 2248 | 2381 | 2006 | 2255 |
| 43 | 2164 | 2603 | 1957 | 2168 | 2059 | 2327 | 1924 | 2061 | 2197 | 2487 | 1963 | 2197 | 2249 | 2379 | 2008 | 2254 |
| 44 | 2164 | 2601 | 1963 | 2175 | 2060 | 2326 | 1924 | 2062 | 2193 | 2483 | 1964 | 2197 | 2249 | 2378 | 2011 | 2255 |
| 45 | 2167 | 2600 | 1963 | 2169 | 2061 | 2319 | 1924 | 2063 | 2196 | 2478 | 1965 | 2199 | 2249 | 2374 | 2013 | 2254 |
| 46 | 2166 | 2595 | 1965 | 2169 | 2061 | 2313 | 1926 | 2061 | 2198 | 2475 | 1967 | 2196 | 2250 | 2372 | 2015 | 2255 |
| 47 | 2169 | 2592 | 2168 | 2168 | 2060 | 2309 | 1923 | 2062 | 2196 | 2470 | 1968 | 2196 | 2251 | 2373 | 2018 | 2256 |
| 48 | 2169 | 2588 | 1968 | 2167 | 2060 | 2304 | 1927 | 2063 | 2195 | 2463 | 1971 | 2196 | 2253 | 2371 | 2022 | 2256 |
| 49 | 2167 | 2588 | 1967 | 2165 | 2061 | 2302 | 1926 | 2060 | 2193 | 2464 | 1972 | 2192 | 2256 | 2370 | 2024 | 2260 |

[symbol]

indicates data missing or illegible when filed

TABLE 2-2

| | | | | | | | | | | | | | | | | |
|---|---|---|---|---|---|---|---|---|---|---|---|---|---|---|---|---|
| 50 | 2167 | 2577 | 1968 | 2164 | 2060 | 2296 | 1928 | 2060 | 2193 | 2459 | 1974 | 2189 | 2255 | 2369 | 2027 | 2262 |
| 51 | 2167 | 2572 | 1968 | 2163 | 2060 | 2290 | 1929 | 2057 | 2194 | 2455 | 1975 | 2184 | 2254 | 2368 | 2028 | 2261 |
| 52 | 2167 | 2562 | 1969 | 2163 | 2062 | 2286 | 1931 | 2058 | 2191 | 2449 | 1976 | 2182 | 2258 | 2366 | 2026 | 2258 |
| 53 | 2166 | 2560 | 1969 | 2162 | 2062 | 2283 | 1930 | 2060 | 2190 | 2446 | 1978 | 2180 | 2259 | 2367 | 2030 | 2263 |
| 54 | 2166 | 2554 | 1969 | 2164 | 2062 | 2278 | 1932 | 2056 | 2189 | 2437 | 1978 | 2177 | 2260 | 2362 | 2031 | 2262 |
| 55 | 2168 | 2545 | 1971 | 2161 | 2061 | 2274 | 1933 | 2058 | 2189 | 2433 | 1981 | 2176 | 2259 | 2357 | 2034 | 2262 |
| 56 | 2167 | 2541 | 1971 | 2164 | 2061 | 2271 | 1933 | 2058 | 2190 | 2430 | 1980 | 2177 | 2258 | 2360 | 2036 | 2216 |
| 57 | 2170 | 2535 | 1971 | 2162 | 2060 | 2266 | 1933 | 2053 | 2189 | 2425 | 1984 | 2175 | 2260 | 2357 | 2035 | 2261 |
| 58 | 2168 | 2527 | 1971 | 2161 | 2062 | 2262 | 1937 | 2054 | 2190 | 2420 | 1986 | 2175 | 2257 | 2352 | 2035 | 2252 |
| 59 | 2172 | 2519 | 1973 | 2160 | 2063 | 2261 | 1936 | 2052 | 2187 | 2418 | 1988 | 2174 | 2260 | 2353 | 2038 | 2260 |
| 60 | 2170 | 2515 | 1975 | 2162 | 2062 | 2256 | 1937 | 2053 | 2187 | 2412 | 1992 | 2172 | 2257 | 2350 | 2041 | 2261 |
| 61 | 2170 | 2512 | 1975 | 2164 | 2062 | 2251 | 1938 | 2052 | 2186 | 2407 | 1992 | 2168 | 2258 | 2350 | 2041 | 2260 |
| 62 | 2171 | 2501 | 1974 | 2162 | 2065 | 2250 | 1938 | 2052 | 2184 | 2485 | 1992 | 2166 | 2258 | 2350 | 2045 | 2261 |
| 63 | 2173 | 2497 | 1976 | 2165 | 2063 | 2246 | 1940 | 2054 | 2184 | 2400 | 1955 | 2166 | 2261 | 2352 | 2049 | 2264 |
| 64 | 2173 | 2491 | 1977 | 2164 | 2063 | 2243 | 1942 | 2052 | 2184 | 2394 | 1995 | 2163 | 2262 | 2349 | 2050 | 2266 |
| 65 | 2173 | 2486 | 1980 | 2165 | 2065 | 2240 | 1940 | 2052 | 2183 | 2391 | 1996 | 2163 | 2266 | 2350 | 2055 | 2267 |
| 66 | 2175 | 2482 | 1979 | 2163 | 2066 | 2231 | 1944 | 2054 | 2183 | 2386 | 1996 | 2162 | 2267 | 2351 | 2058 | 2270 |
| 67 | 2174 | 2475 | 1979 | 2161 | 2066 | 2231 | 1942 | 2054 | 2182 | 2379 | 1996 | 2159 | 2269 | 2349 | 2060 | 2272 |
| 68 | 2175 | 2470 | 1979 | 2159 | 2089 | 2231 | 1941 | 2052 | 2181 | 2377 | 1997 | 2159 | 2269 | 2351 | 2063 | 2273 |
| 69 | 2176 | 2467 | 1982 | 2161 | 2067 | 2228 | 1946 | 2053 | 2180 | 2372 | 1995 | 2153 | 2271 | 2349 | 2067 | 2276 |
| 70 | 2177 | 2463 | 1985 | 2060 | 2070 | 2226 | 1948 | 2055 | 2178 | 2364 | 1993 | 2151 | 2275 | 2347 | 2068 | 2276 |
| 71 | 2176 | 2456 | 1983 | 2163 | 2068 | 2224 | 1949 | 2054 | 2177 | 2363 | 1994 | 2148 | 2275 | 2347 | 2070 | 2279 |
| 72 | 2177 | 2452 | 1984 | 2158 | 2070 | 2222 | 1951 | 2055 | 2177 | 2358 | 1994 | 2147 | 2276 | 2344 | 2072 | 2281 |
| 73 | 2176 | 2446 | 1983 | 2155 | 2073 | 2219 | 1951 | 2054 | 2177 | 2352 | 1994 | 2144 | 2275 | 2343 | 2073 | 2282 |
| 74 | 2175 | 2440 | 1988 | 2154 | 2074 | 2216 | 1952 | 2057 | 2177 | 2351 | 1995 | 2144 | 2279 | 2342 | 2076 | 2278 |
| 75 | 2177 | 2437 | 1987 | 2156 | 2074 | 2214 | 1954 | 2057 | 2177 | 2346 | 1995 | 2143 | 2282 | 2344 | 2080 | 2286 |
| 76 | 2177 | 2430 | 1990 | 2154 | 2075 | 2210 | 1956 | 2060 | 2177 | 2343 | 1996 | 2143 | 2284 | 2343 | 2081 | 2289 |
| 77 | 2178 | 2424 | 1989 | 2155 | 2078 | 2210 | 1954 | 2060 | 2180 | 2343 | 1995 | 2143 | 2286 | 2343 | 2086 | 2291 |
| 78 | 2179 | 2419 | 1991 | 2154 | 2080 | 2207 | 1959 | 2061 | 2176 | 2338 | 1998 | 2144 | 2288 | 2344 | 2089 | 2293 |
| 79 | 2176 | 2411 | 1991 | 2163 | 2079 | 2206 | 1962 | 2063 | 2176 | 2335 | 1998 | 2147 | 2290 | 2345 | 2099 | 2295 |
| 80 | 2177 | 2407 | 1989 | 2155 | 2082 | 2206 | 1961 | 2064 | 2176 | 2334 | 1998 | 2148 | 2290 | 2344 | 2094 | 2294 |
| 81 | 2177 | 2404 | 1991 | 2156 | 2081 | 2203 | 1963 | 2065 | 2176 | 2332 | 1999 | 2146 | 2291 | 2345 | 2094 | 2295 |
| 82 | 2181 | 2399 | 1992 | 2153 | 2080 | 2200 | 1965 | 2066 | 2173 | 2327 | 1999 | 2145 | 2293 | 2344 | 2097 | 2294 |
| 83 | 2175 | 2394 | 1990 | 2153 | 2084 | 2195 | 1965 | 2068 | 2172 | 2325 | 2000 | 2145 | 2295 | 2345 | 2100 | 2292 |
| 84 | 2175 | 2396 | 1993 | 2153 | 2085 | 2199 | 1965 | 2069 | 2172 | 2327 | 1999 | 2144 | 2295 | 2347 | 2101 | 2294 |
| 85 | 2177 | 2387 | 1993 | 2153 | 2087 | 2195 | 1969 | 2069 | 2172 | 2319 | 2001 | 2143 | 2297 | 2347 | 2103 | 2293/ |
| 86 | 2177 | 2388 | 1996 | 2155 | 2086 | 2197 | 1969 | 2071 | 2172 | 2318 | 1999 | 2148 | 2300 | 2348 | 2107 | 2293 |
| 87 | 2182 | 2383 | 1997 | 2154 | 2087 | 2193 | 1971 | 2073 | 2171 | 2315 | 2002 | 2146 | 2302 | 2350 | 2111 | 2295 |
| 88 | 2179 | 2381 | 1996 | 2155 | 2088 | 2194 | 1971 | 2074 | 2169 | 2317 | 2002 | 2147 | 2305 | 2352 | 2114 | 2295 |
| 89 | 2183 | 2378 | 2000 | 2157 | 2091 | 2192 | 1975 | 2075 | 2173 | 2316 | 2003 | 2148 | 2308 | 2352 | 2117 | 2294 |
| 90 | 2181 | 2375 | 2002 | 2161 | 2092 | 2191 | 1977 | 2076 | 2173 | 2314 | 2009 | 2147 | 2310 | 2352 | 2122 | 2295 |
| 91 | 2181 | 2373 | 2002 | 2156 | 2093 | 2192 | 1979 | 2078 | 2173 | 2310 | 2004 | 2147 | 2312 | 2354 | 2124 | 2298 |
| 92 | 2184 | 2373 | 2005 | 2158 | 2096 | 2192 | 1979 | 2081 | 2167 | 2309 | 1999 | 2148 | 2313 | 2354 | 2126 | 2297 |
| 93 | 2184 | 2372 | 2007 | 2161 | 2095 | 2188 | 1981 | 2081 | 2170 | 2307 | 2005 | 2144 | 2316 | 2355 | 2134 | 2299 |
| 94 | 2187 | 2371 | 2006 | 2158 | 2100 | 2188 | 1985 | 2082 | 2168 | 2304 | 2006 | 2146 | 2317 | 2355 | 2133 | 2302 |
| 95 | 2187 | 2365 | 2009 | 2160 | 2099 | 2187 | 1984 | 2079 | 2168 | 2300 | 2006 | 2145 | 2319 | 2358 | 2139 | 2302 |
| 96 | 2187 | 2364 | 2008 | 2158 | 2101 | 2185 | 1987 | 2081 | 2167 | 2299 | 2006 | 2142 | 2321 | 2359 | 2142 | 2303 |
| 97 | 2188 | 2362 | 2009 | 2160 | 2103 | 2186 | 1989 | 2086 | 2166 | 2254 | 2007 | 2142 | 2323 | 2357 | 2145 | 2307 |
| 98 | 2190 | 2360 | 2012 | 2162 | 2105 | 2186 | 1991 | 2084 | 2166 | 2296 | 2007 | 2144 | 2325 | 2361 | 2149 | 2307 |
| 99 | 2192 | 2359 | 2013 | 2162 | 2105 | 2185 | 1989 | 2089 | 2165 | 2293 | 2007 | 2143 | 2327 | 2362 | 2151 | 2310 |

In addition, as shown in FIGS. 11 and 12, and Tables 3-1 and 3-1, it may be seen that a relative value may be used to analyze and determine the pattern because the different tips 61 have the measurement values slightly different from each other, but show the same pattern when analyzing whether the sample is the whole blood or the blood plasma or analyzing the measurement value of the whole blood or blood plasma when the whole blood or the blood plasma is not absorbed by comparing the blood non-absorption data with the blood absorption data obtained from the different two tips used for one measurement by using the same reflective photosensor 80, in the automatic immunoassay device including the tip which may be moved up, down, left and right based on the round cartridge according to an exemplary embodiment of the present invention.

TABLE 3-1

| Tip1 | | | | | Tip 2 | | | |
|---|---|---|---|---|---|---|---|---|
| | [symbol] | | | | [symbol] | | | |
| | tip | plsam | wb | tip2 | tip | plsam | wb | tip2 |
| 0 | 2066 | 2038 | 2018 | 2068 | 2104 | 2074 | 2061 | 2104 |
| 1 | 2067 | 2035 | 2018 | 2068 | 2105 | 2074 | 2062 | 2103 |
| 2 | 2071 | 2040 | 2016 | 2065 | 2103 | 2073 | 2059 | 2103 |
| 3 | 2068 | 2039 | 2023 | 2067 | 2107 | 2074 | 2061 | 2103 |
| 4 | 2069 | 2038 | 2015 | 2067 | 2104 | 2073 | 2063 | 2106 |
| 5 | 2068 | 2037 | 2015 | 2066 | 2104 | 2072 | 2059 | 2104 |
| 6 | 2067 | 2037 | 2009 | 2065 | 2106 | 2071 | 2057 | 2105 |
| 7 | 2069 | 2033 | 2001 | 2066 | 2105 | 2071 | 2056 | 2103 |
| 8 | 2067 | 2029 | 1996 | 2067 | 2104 | 2070 | 2050 | 2107 |
| 9 | 2067 | 2024 | 1986 | 2066 | 2101 | 2068 | 2041 | 2104 |
| 10 | 2068 | 2021 | 1969 | 2067 | 2105 | 2064 | 2036 | 2102 |
| 11 | 2069 | 2014 | 1962 | 2066 | 2107 | 2065 | 2026 | 2104 |
| 12 | 2069 | 2007 | 1945 | 2065 | 2106 | 2057 | 2014 | 2106 |
| 13 | 2068 | 2007 | 1928 | 2064 | 2107 | 2054 | 2000 | 2105 |
| 14 | 2067 | 2006 | 1918 | 2066 | 2111 | 2051 | 1988 | 2105 |
| 15 | 2066 | 2013 | 1906 | 2067 | 2109 | 2045 | 1980 | 2107 |
| 16 | 2057 | 2017 | 1892 | 2063 | 2113 | 2041 | 1963 | 2109 |
| 17 | 2068 | 2033 | 1886 | 2066 | 2111 | 2042 | 1957 | 2111 |
| 18 | 2066 | 2060 | 1874 | 2063 | 2113 | 2041 | 1944 | 2109 |
| 19 | 2065 | 2072 | 1865 | 2065 | 2114 | 2046 | 1993 | 2113 |
| 20 | 2066 | 2103 | 1861 | 2066 | 2113 | 2052 | 1921 | 2110 |
| 21 | 2067 | 2137 | 1851 | 2064 | 2113 | 2061 | 1916 | 2110 |
| 22 | 2065 | 2154 | 1845 | 2066 | 2111 | 2079 | 1904 | 2109 |
| 23 | 2072 | 2190 | 1840 | 2062 | 2110 | 2089 | 1991 | 2105 |
| 24 | 2067 | 2226 | 1836 | 2063 | 2107 | 2110 | 1890 | 2107 |
| 25 | 2067 | 2246 | 1826 | 2063 | 2109 | 2131 | 1884 | 2103 |
| 26 | 2066 | 2280 | 1826 | 2066 | 2109 | 2144 | 1882 | 2104 |
| 27 | 2068 | 2312 | 1822 | 2067 | 2110 | 2166 | 1873 | 2102 |
| 28 | 2068 | 2328 | 1818 | 2068 | 2106 | 2189 | 1870 | 2104 |
| 29 | 2072 | 2355 | 1815 | 2069 | 2108 | 2202 | 1867 | 2104 |
| 30 | 2073 | 2375 | 1814 | 2071 | 2112 | 2224 | 1864 | 2100 |
| 31 | 2073 | 2386 | 1812 | 2072 | 2111 | 2242 | 1864 | 2106 |
| 32 | 2079 | 2397 | 1811 | 2071 | 2113 | 2251 | 1866 | 2105 |
| 33 | 2076 | 2406 | 1810 | 2072 | 2114 | 2263 | 1864 | 2105 |
| 34 | 2075 | 2410 | 1812 | 2072 | 2115 | 2271 | 1864 | 2105 |
| 35 | 2077 | 2414 | 1812 | 2074 | 2117 | 2275 | 1868 | 2108 |
| 36 | 2077 | 2416 | 1811 | 2071 | 2118 | 2281 | 1866 | 2107 |
| 37 | 2079 | 2419 | 1815 | 2072 | 2119 | 2285 | 1868 | 2110 |
| 38 | 2079 | 2416 | 1814 | 2072 | 2116 | 2287 | 1872 | 2108 |
| 39 | 2081 | 2416 | 1815 | 2073 | 2121 | 2290 | 1870 | 2109 |
| 40 | 2082 | 2413 | 1817 | 2074 | 2127 | 2296 | 1873 | 2109 |
| 41 | 2082 | 2413 | 1817 | 2072 | 2127 | 2294 | 1876 | 2114 |
| 42 | 2082 | 2407 | 1819 | 2073 | 2130 | 2293 | 1879 | 2112 |
| 43 | 2082 | 2409 | 1821 | 2076 | 2132 | 2293 | 1884 | 2114 |
| 44 | 2084 | 2403 | 1823 | 2073 | 2132 | 2294 | 1883 | 2116 |
| 45 | 2086 | 2404 | 1824 | 2076 | 2136 | 2296 | 1882 | 2117 |
| 46 | 2086 | 2399 | 1827 | 2077 | 2138 | 2295 | 1889 | 2119 |
| 47 | 2089 | 2394 | 1830 | 2076 | 2141 | 2292 | 1891 | 2122 |
| 48 | 2092 | 2392 | 1830 | 2079 | 2142 | 2295 | 1895 | 2125 |
| 49 | 2092 | 2391 | 1832 | 2083 | 2146 | 2295 | 1900 | 2125 |
| 50 | 2097 | 2386 | 1832 | 2080 | 2147 | 2297 | 1901 | 2127 |

TABLE 3-2

| | | | | | | | | |
|---|---|---|---|---|---|---|---|---|
| 51 | 2095 | 2336 | 1835 | 2079 | 2150 | 2293 | 1904 | 2130 |
| 52 | 2097 | 2383 | 1836 | 2082 | 2153 | 2296 | 1908 | 2131 |
| 53 | 2096 | 2382 | 1839 | 2080 | 2158 | 2296 | 1910 | 2137 |
| 54 | 2099 | 2377 | 1840 | 2080 | 2159 | 2296 | 1914 | 2138 |
| 55 | 2097 | 2378 | 1836 | 2082 | 2166 | 2294 | 1918 | 2140 |
| 56 | 2098 | 2372 | 1843 | 2082 | 2167 | 2296 | 1917 | 2144 |
| 57 | 2098 | 2368 | 1845 | 2084 | 2173 | 2295 | 1925 | 2148 |
| 58 | 2101 | 2355 | 1849 | 2085 | 2175 | 2294 | 1932 | 2151 |
| 59 | 2106 | 2363 | 1851 | 2087 | 2182 | 2296 | 1936 | 2156 |
| 60 | 2108 | 2359 | 1856 | 2088 | 2183 | 2295 | 1939 | 2158 |
| 61 | 2112 | 2356 | 1859 | 2091 | 2188 | 2294 | 1945 | 2162 |
| 62 | 2112 | 2352 | 1859 | 2093 | 2194 | 2294 | 1948 | 2168 |
| 63 | 2118 | 2350 | 1863 | 2096 | 2196 | 2293 | 1956 | 2172 |
| 64 | 2119 | 2346 | 1868 | 2097 | 2201 | 2294 | 1960 | 2172 |
| 65 | 2125 | 2340 | 1871 | 2099 | 2210 | 2293 | 1964 | 2179 |
| 66 | 2127 | 2339 | 1874 | 2101 | 2208 | 2294 | 1970 | 2186 |
| 67 | 2129 | 2346 | 1877 | 2105 | 2215 | 2293 | 1974 | 2188 |
| 68 | 2134 | 2335 | 1879 | 2107 | 2218 | 2294 | 1977 | 2188 |
| 69 | 2139 | 2334 | 1884 | 2109 | 2222 | 2293 | 1983 | 2193 |
| 70 | 2137 | 2334 | 1886 | 2112 | 2224 | 2294 | 1989 | 2194 |
| 71 | 2143 | 2330 | 1889 | 2110 | 2230 | 2295 | 1991 | 2199 |
| 72 | 2146 | 2327 | 1894 | 2117 | 2233 | 2293 | 1996 | 2202 |
| 73 | 2146 | 2327 | 1895 | 2119 | 2236 | 2292 | 2001 | 2205 |
| 74 | 2150 | 2328 | 1895 | 2119 | 2242 | 2293 | 2005 | 2207 |
| 75 | 2152 | 2322 | 1900 | 2121 | 2241 | 2295 | 2007 | 2213 |
| 76 | 2153 | 2323 | 1905 | 2122 | 2244 | 2294 | 2015 | 2214 |
| 77 | 2158 | 2318 | 1906 | 2125 | 2251 | 2297 | 2016 | 2219 |
| 78 | 2159 | 2319 | 1912 | 2125 | 2253 | 2296 | 2023 | 2221 |
| 79 | 2160 | 2317 | 1912 | 2130 | 2256 | 2296 | 2030 | 2226 |
| 80 | 2165 | 2316 | 1915 | 2130 | 2262 | 2298 | 2029 | 2230 |
| 81 | 2166 | 2314 | 1917 | 2132 | 2268 | 2297 | 2036 | 2234 |
| 82 | 2169 | 2316 | 1923 | 2136 | 2273 | 2301 | 2043 | 2236 |
| 83 | 2174 | 2312 | 1926 | 2137 | 2277 | 2302 | 2048 | 2241 |
| 84 | 2179 | 2313 | 1932 | 2142 | 2281 | 2302 | 2054 | 2247 |
| 85 | 2183 | 2309 | 1936 | 2145 | 2287 | 2306 | 2062 | 2247 |
| 86 | 2183 | 2307 | 1937 | 2146 | 2293 | 2310 | 2066 | 2255 |
| 87 | 2188 | 2306 | 1944 | 2152 | 2294 | 2308 | 2074 | 2260 |
| 88 | 2189 | 2307 | 1948 | 2154 | 2304 | 2312 | 2083 | 2266 |
| 89 | 2194 | 2303 | 1951 | 2155 | 2309 | 2313 | 2084 | 2271 |
| 90 | 2200 | 2304 | 1959 | 2164 | 2313 | 2316 | 2092 | 2274 |
| 91 | 2203 | 2305 | 1963 | 2169 | 2321 | 2319 | 2098 | 2280 |
| 92 | 2206 | 2309 | 1965 | 2170 | 2324 | 2323 | 2102 | 2286 |
| 93 | 2211 | 2308 | 1971 | 2177 | 2329 | 2320 | 2108 | 2295 |
| 94 | 2215 | 2305 | 1977 | 2177 | 2337 | 2324 | 2115 | 2296 |
| 95 | 2217 | 2301 | 1973 | 2179 | 2339 | 2328 | 2119 | 2605 |
| 96 | 2224 | 2307 | 1985 | 2182 | 2341 | 2330 | 2124 | 2310 |
| 97 | 2224 | 2307 | 1987 | 2185 | 2347 | 2332 | 2131 | 2311 |
| 98 | 2231 | 2304 | 1998 | 2186 | 2351 | 2334 | 2133 | 2316 |
| 99 | 2233 | 2306 | 1995 | 2190 | 2353 | 2336 | 2143 | 2320 |

### INDUSTRIAL APPLICABILITY

The method of automatically distinguishing the absorption or non-absorption of the whole blood or the blood plasma by using the reflective photosensor according to an exemplary embodiment of the present invention may accurately check whether the whole blood or the blood plasma is absorbed or whether the whole blood or the blood plasma is not absorbed from the round cartridge into the pipette mounted on the tip of the automatic immunoassay device including the round cartridge which may simultaneously perform the centrifugation and automatic analysis of the blood sample and the tip which may be moved up, down, left and right based on the round cartridge.

1. A method of automatically distinguishing absorption or non-absorption of whole blood or blood plasma by using a reflective photosensor in an automatic immunoassay device including a round cartridge which may simultaneously perform the centrifugation and automatic analysis of a blood sample and a tip which may be moved up, down, left and right based on the round cartridge, the method comprising:
installing the reflective photosensor below the round cartridge;
mounting the tip above the round cartridge, and continuously measuring blood non-absorption data in a range including a position where the blood is absorbed by using the reflective photosensor, collecting the measured data and storing the collected data while the tip is raised;
continuously measuring blood absorption data by using the reflective photosensor while the tip is lowered and absorbs the blood present in the range including the position where the blood is absorbed from the round cartridge; and
determining whether a type of the blood is whole blood or blood plasma, or whether the whole blood or the blood plasma is not absorbed by comparing the blood non-absorption data with the blood absorption data.

2. The method of claim 1, wherein the reflective photosensor includes a light source which is a long and thin light emitting diode (LED) disposed below the round cartridge in an axial direction of the cartridge, and a light receiving element which is a charge coupled device (CCD) linear image sensor opposite to the light source, and installed in one row below the cartridge in the axial direction of the cartridge.

3. The method of claim 1, wherein
the determining of whether the type of the blood is the whole blood or the blood plasma, or whether the whole blood or the blood plasma is not absorbed by comparing the blood non-absorption data with the blood absorption data includes performing the measurements of whether the whole blood or the blood plasma is not absorbed into the tip or at least two or more different tips, whether the whole blood is absorbed into the tip, and whether the blood plasma is absorbed into the tip.

4. The method of claim 3, wherein
in the determining of whether the type of the blood is the whole blood or the blood plasma, or whether the whole blood or the blood plasma is not absorbed by comparing the blood non-absorption data with the blood absorption data, the determination is made by a slope, which is a ratio of the measurement value of the whole blood or blood plasma obtained by using the reflective photosensor to a height of the tip.

5. The method of claim 4, wherein
in the determining of whether the type of the blood is the whole blood or the blood plasma, or whether the whole blood or the blood plasma is not absorbed by comparing the blood non-absorption data with the blood absorption data,
the determination is made by the slope of data obtained from the continuous measurements of the blood in a section including
a point at which a difference between values obtained from the continuous measurements of the whole blood (wb) and the (blood) plasma by using the reflective photosensor begins to occur and
a point at which the slope of the values obtained from the continuous measurements of the whole blood (wb) and the (blood) plasma by using the reflective photosensor has a maximum value.

6. The method of claim 5, wherein
the tip in the section has a height of 10 steps or more and 46 steps or less when a total height is 200 steps.

7. The method of claim 5, wherein
500 to 700 is a maximum value of a difference between the measurement values of the whole blood and the blood plasma obtained by using the reflective photosensor.
